(19) United States
(12) Patent Application Publication
(10) Pub. No.: US 2026/0115342 A1
(43) Pub. Date: Apr. 30, 2026

(54) Tub Ultraviolet Treatment

(71) Applicant: Sensor Electronic Technology, Inc., Columbia, SC (US)

(72) Inventor: Piyush Sharma, Columbia, SC (US)

(21) Appl. No.: 19/367,879

(22) Filed: Oct. 24, 2025

Related U.S. Application Data
(60) Provisional application No. 63/711,298, filed on Oct. 24, 2024.

(51) Int. Cl. A61L 2/10; A47K 3/38; A61L 2/24

(52) U.S. Cl. CPC A61L 2/10; A47K 3/38; A61L 2/24; A61L 2202/11; A61L 2202/14

(57) ABSTRACT

A shower curtain with a plurality of ultraviolet light sources embedded between first and second liner curtains. Each liner curtain can be fabricated from a water-resistant material and can be secured to the other liner curtain. Each ultraviolet light source can be configured to emit ultraviolet light through a corresponding location of at least one of the first or second liner curtains. One or both liner curtains can be fabricated from ultraviolet transparent material. An ultraviolet treatment system can include the shower curtain and a power and control component configured to operate the ultraviolet light sources to treat at least one surface located around the shower curtain.

### REFERENCE TO RELATED APPLICATIONS

The current application claims the benefit of U.S. Provisional Application 63/711,298, filed on 24 Oct. 2024, which is hereby incorporated by reference.

### TECHNICAL FIELD

The disclosure relates generally to treating a bathtub or shower with ultraviolet light, and more particularly, to a solution that utilizes ultraviolet light to suppress or prevent growth of mold and other microorganisms in a bathtub or shower environment.

### BACKGROUND ART

The prevailing approaches for bathroom mold prevention encompass the use of various cleaning agents, ventilation systems, and dehumidifiers. Despite their widespread use, these approaches have certain challenges. For example, they are frequently labor-intensive, demanding substantial effort for application, and necessitate frequent maintenance. Moreover, these approaches may fall short in addressing the root cause of mold formation in challenging spaces, such as the bathtub and shower curtain, where conventional methods often prove inadequate.

One significant problem with the existing state of the art lies in the absence of a proactive and automated approach to tackle the issue of mold prevention. Traditional approaches often rely on manual cleaning practices or passive ventilation systems, resulting in the growth of mold in overlooked areas. The reliance on these approaches also may lead to gaps in continuous protection, particularly during periods of inactivity. Consequently, mold can develop over time, undermining the effectiveness of the prevailing mold prevention strategies.

While alternative approaches such as improved cleaning agents or advanced ventilation systems exist, they may not comprehensively address the challenges posed by mold-prone areas.

The drawbacks associated with existing alternatives further emphasize the need for innovation in the field of mold prevention. Labor-intensive application of cleaning agents, the reliance on passive ventilation systems with limited targeting capabilities, and the insufficient coverage of challenging areas underscore the limitations of current alternatives.

### SUMMARY OF THE INVENTION

In response to these challenges, embodiments of the invention provide a solution which incorporates ultraviolet (UV) light sources, such as UV light emitting devices (LEDs), within the bathroom environment. In embodiments, UV LEDs are embedded between two plastic liner curtains for a bathtub or shower to provide mold prevention. More particular embodiments utilize ultraviolet C (UV-C) light to target and eliminate microorganisms, thereby preventing or suppressing mold formation.

To enhance its functionality and address the limitations of the current state of the art, embodiments of the invention can include a real-time control system. This system, which can receive input from sensors, such as motion and light sensors, can provide safe and efficient operation of the UV sources, thereby providing a comprehensive and intelligent solution to mold prevention in bathrooms.

Aspects of the invention provide a shower curtain with a plurality of ultraviolet light sources embedded between first and second liner curtains. Each liner curtain can be fabricated from a water-resistant material and can be secured to the other liner curtain. Each ultraviolet light source can be configured to emit ultraviolet light through a corresponding location of at least one of the first or second liner curtains. One or both liner curtains can be fabricated from ultraviolet transparent material. An ultraviolet treatment system can include the shower curtain and a power and control component configured to operate the ultraviolet light sources to treat at least one surface located around the shower curtain.

A first aspect of the invention provides a shower curtain comprising: a first liner curtain fabricated from a first water-resistant material; a second liner curtain fabricated from second water-resistant material, wherein the second liner curtain is secured to the first liner curtain; and a plurality of ultraviolet light sources embedded between the first and second liner curtains, wherein at least one of the first water-resistant material or the second water-resistant material is an ultraviolet transparent material, and wherein each of the plurality of ultraviolet light sources is configured to emit ultraviolet light through a corresponding location of the ultraviolet transparent material.

A second aspect of the invention provides an ultraviolet treatment system comprising: a power and control component; and a shower curtain, the shower curtain comprising: at least one interface configured for selective connection to the power and control component; a first liner curtain fabricated from a first water-resistant material; a second liner curtain fabricated from second water-resistant material, wherein the second liner curtain is secured to the first liner curtain; a plurality of ultraviolet light sources embedded between the first and second liner curtains; and a plurality of connectivity structures embedded between the first liner curtain and the second liner curtain, wherein each connectivity structure connects at least one of the plurality of ultraviolet light sources to the at least one interface, and wherein each of the plurality of ultraviolet light sources is configured to emit ultraviolet light through a location of at least one of the first liner curtain or the second liner curtain, and wherein the power and control component is configured to operate the ultraviolet light sources to treat at least one surface located around the shower curtain.

A third aspect of the invention provides an ultraviolet treatment system comprising: a power and control component comprising: at least one computing unit; and at least one output device configured to provide information to a user regarding an operating status of the ultraviolet treatment system; and a shower curtain, the shower curtain comprising: at least one interface configured for selective connection to the power and control component; a first liner curtain fabricated from a first water-resistant material; a second liner curtain fabricated from second water-resistant material, wherein the second liner curtain is secured to the first liner curtain; a plurality of ultraviolet light sources embedded between the first and second liner curtains; and a plurality of connectivity structures embedded between the first liner curtain and the second liner curtain, wherein each connectivity structure connects at least one of the plurality of ultraviolet light sources to the at least one interface, and wherein each of the plurality of ultraviolet light sources is configured to emit ultraviolet light through a location of at least one of the first liner curtain or the second liner curtain; and wherein the at least one computing unit is configured to operate the ultraviolet light sources to treat at least one surface located around the shower curtain.

The illustrative aspects of the invention are designed to solve one or more of the problems herein described and/or one or more other problems not discussed.

### BRIEF DESCRIPTION OF THE DRAWINGS

These and other features of the disclosure will be more readily understood from the following detailed description of the various aspects of the invention taken in conjunction with the accompanying drawings that depict various aspects of the invention.

FIG. 1 shows an illustrative bathtub and shower curtain according to the prior art.

FIG. 2 shows an illustrative bathtub and shower curtain according to embodiments.

FIGS. 3A and 3B show side and partial front views of an illustrative ultraviolet treatment system according to embodiments.

FIG. 4 shows a schematic of an illustrative treatment system according to an embodiment.

FIG. 5 shows an illustrative process for treating an area around a shower curtain according to embodiments.

FIG. 6 shows an illustrative process for handling an error condition according to embodiments.

It is noted that the drawings may not be to scale. The drawings are intended to depict only typical aspects of the invention, and therefore should not be considered as limiting the scope of the invention. In the drawings, like numbering represents like elements between the drawings.

### DETAILED DESCRIPTION OF THE INVENTION

As discussed herein, embodiments provide a shower curtain that includes ultraviolet light sources. The shower curtain can be mounted near a bathtub, a shower stall, and/or the like, collectively referred to herein as a tub, and be configured to emit ultraviolet light that is directed onto various surfaces near the shower curtain, including surfaces of the tub.

Embodiments of the ultraviolet light can include UV-C light, which is well-documented for its germicidal properties, effectively killing bacteria, viruses, and other pathogens upon exposure. By emitting the ultraviolet light onto a surface, such as the tub or one or more walls around the tub, embodiments can effectively eliminate bacterial contamination, thus ensuring improved hygiene and user safety. Embodiments of the ultraviolet light can be used in conjunction with a photocatalyst to provide disinfection.

Embodiments incorporate a matrix of ultraviolet light emitting sources into a shower curtain. In embodiments, the sources can include UV LEDs, which can be energy-efficient and long-lasting, ensuring reliable performance and minimal maintenance requirements. The UV sources can be positioned within the shower curtain, and can be configured to emit, for example, short-wavelength UV-C light, known for its germicidal properties. This UV-C light effectively destroys the DNA of microorganisms, including mold spores, bacteria, and viruses, thereby preventing their proliferation and ensuring a hygienic environment within the tub area.

Embodiments can provide regular, automated treatment (e.g., disinfection) of the tub area without the need for manual intervention. Such an area is susceptible to standing water, which can promote the growth of microorganisms over time. Unlike traditional approaches, such as chemical treatments or surface coatings, which may offer temporary relief but require frequent reapplication and maintenance, embodiments can include an ultraviolet LED illumination component which can provide ongoing protection against microbial contamination, which can ensure long-term hygiene.

Embodiments can be powered by a dedicated driver and controlled by a microcontroller to ensure effective disinfection. Embodiments also can include one or more sensors, which can acquire data used to operate the ultraviolet illumination, thereby enhancing the safety and usability of the curtain. These sensors, which are communicatively connected to the microcontroller, can increase user safety and minimize the risk of UV-related health hazards. A non-exhaustive list of sensors includes proximity sensors, such as light and/or motion detectors, which can detect the presence of users in the vicinity of the tub. When human presence is detected, embodiments can automatically deactivate the UV LEDs, which can prevent any potential inadvertent exposure to UV-C radiation. This safety feature mitigates the risk of UV-related health hazards. Additional sensor configurations, such as incorporating light sensors, temperature sensors, moisture sensors, and/or the like, may be utilized to further augment safety measures and/or add other functionality to the system.

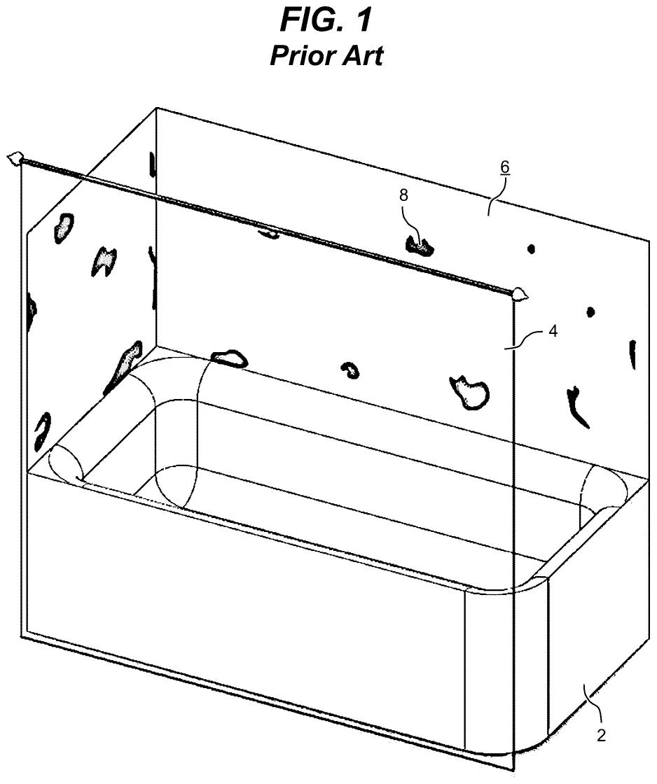

Turning to the drawings, FIG. 1 shows an illustrative bathtub 2 and shower curtain 4 according to the prior art. As is known, the shower curtain 4 is utilized to prevent water from leaving the tub area, e.g., when an individual is taking a shower, while also allowing an individual to readily enter and exit the bathtub 2. As illustrated, due to repeated exposure to water and standing water remaining after use, mold 8 can grow on one or more walls 6 around the bathtub 2 and/or surfaces of the bathtub 2 itself. Such growth also can occur on the shower curtain 4, particularly a bottom portion that can be located partially within the bathtub 2.

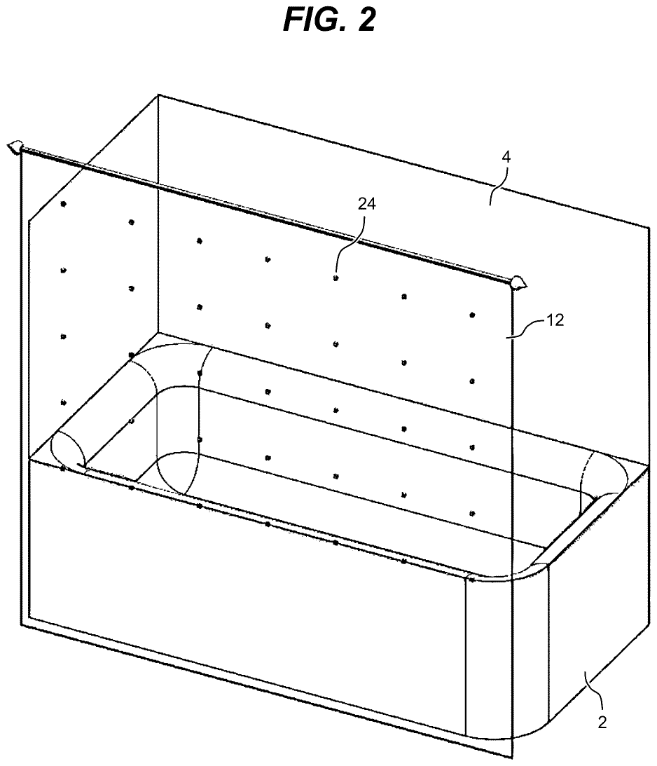

FIG. 2 shows an illustrative bathtub 2 and shower curtain 12 according to embodiments. As illustrated, the shower curtain 12 includes a plurality of ultraviolet light sources 24 embedded therein. Each ultraviolet light source 24 can be configured to emit ultraviolet radiation therefrom, which can be directed within the tub area, e.g., toward the bathtub 2 and one or more walls 4 partially surrounding the bathtub 2.

While not separately illustrated for clarity, it is understood that embodiments can be utilized in conjunction with any of various types of bathtubs as well as other environments in which flowing water is used, but may not include a bathtub. For example, embodiments can be used in conjunction with curtains used to selectively enclose an opening of a shower or shower stall.

In embodiments, the ultraviolet light sources 24 emit light within one or more subsets of the ultraviolet light range of wavelengths. In embodiments, the ultraviolet light comprises ultraviolet-C light, which has a peak wavelength in a range between 100 nanometers and 280 nanometers. In this case, the ultraviolet light can be configured to kill or inactivate microorganisms that may be present on the bathtub 2, walls 4, outer surface of the curtain 12, etc. In embodiments, the ultraviolet light comprises ultraviolet-A light, which has a peak wavelength in a range between 315 nanometers and 400 nanometers. Such ultraviolet light can be used to suppress growth, activate a photocatalyst, and/or the like.

In embodiments, the ultraviolet light sources 24 can be configured to selectively emit, concurrently and/or at different times, ultraviolet radiation within two or more subsets of the ultraviolet light range of wavelengths. In embodiments, the ultraviolet light sources 24 can be manually operated, operated by a computing unit, operated in response to sensor data, and/or the like.

In embodiments, each ultraviolet light source 24 can comprise an ultraviolet light emitting diode. In other embodiments, some or all of the ultraviolet light sources 24 can comprise a termination point of a light guiding structure, such as an optical fiber and/or the like, embedded within the curtain 12 and from which ultraviolet light can be emitted. To this extent, a curtain 12 described herein can include any of various configurations and/or combinations of ultraviolet light emitting diodes and/or light guiding structures through which ultraviolet light travels and from which ultraviolet light is emitted.

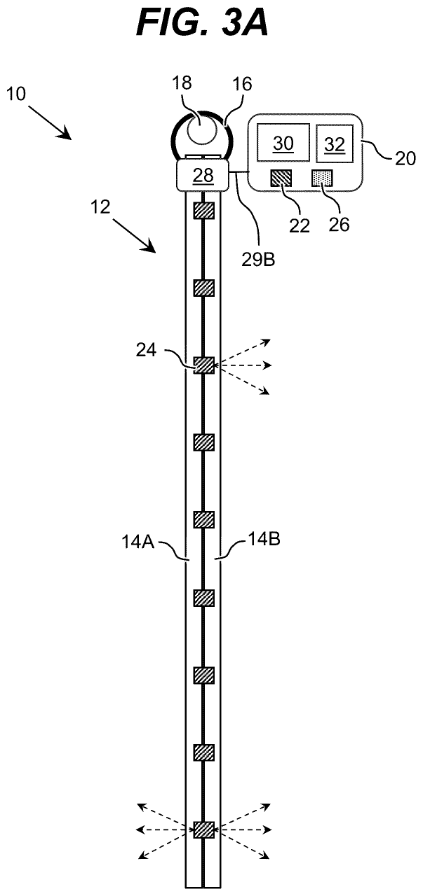

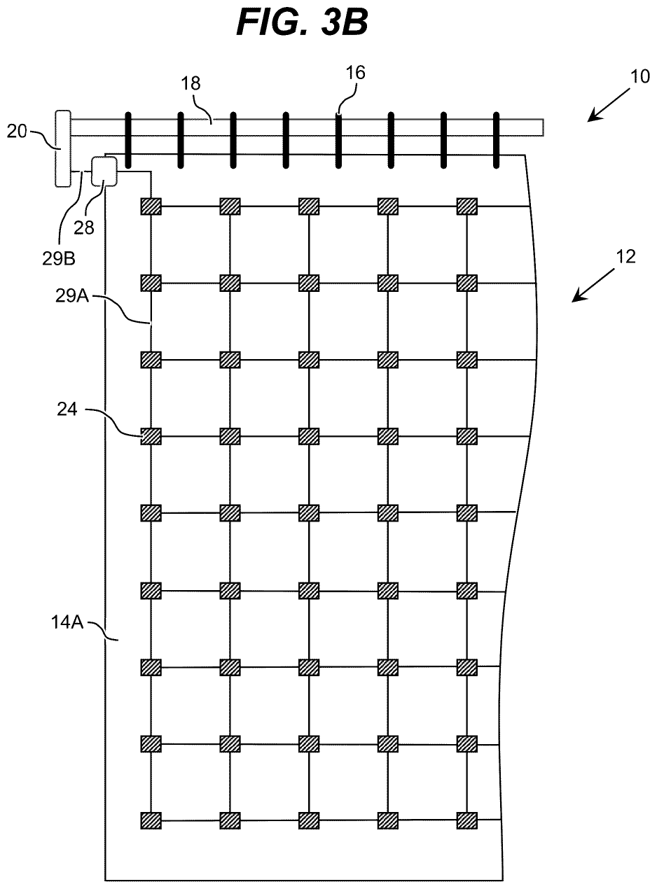

FIGS. 3A and 3B show side and partial front views of an illustrative ultraviolet treatment system 10 according to embodiments. As illustrated, the ultraviolet treatment system 10 can include a shower curtain 12, which can be supported by a rod 18 that can be mounted to walls on opposing sides of a bathtub 2. The shower curtain 12 can be supported by the rod 18 using a solution that allows the shower curtain 12 to be selectively opened and closed to allow an individual to enter and exit the bathtub 2. For example, as illustrated, the shower curtain 12 can include a plurality of rings 16, which can be supported by the rod 18 and routed through an opening in the shower curtain 12. However, it is understood that the rod 18 and rings 16 are only illustrative of numerous solutions that can be utilized to support the shower curtain 12 in a manner that allows for its selective opening and closing in embodiments.

As illustrated in FIG. 3A, the shower curtain 12 can comprise two liner curtains 14A, 14B with the ultraviolet light sources 24 embedded therein. In embodiments, one or both liner curtains 14A, 14B are fabricated from an ultraviolet transparent material. The liner curtains 14A, 14B can be fabricated from durable and water-resistant materials. In embodiments, a liner curtain 14A, 14B can be fabricated and/or coated with a mold and/or mildew-resistant material. In more particular embodiments, the liner curtains 14A, 14B are fabricated from a plastic material. Illustrative materials include ethylene vinyl acetate (EVA), poly ethylene vinyl acetate (PEVA), polycarbonate, acrylic (Poly (methyl methacrylate) or PMMA), various grades of glass designed for ultraviolet transmission, and/or the like.

In embodiments, the liner curtains 14A, 14B are permanently secured to one another. The liner curtains 14A, 14B can be secured to each other using any of various solutions, including heat sealing, applying an adhesive, and/or the like.

In embodiments, the liner curtains 14A, 14B can have a thickness that provides adequate durability and flexibility, while ensuring effective ultraviolet light transmission to eliminate mold and micro-organisms. In embodiments, the thickness is in a range from approximately 0.5 millimeters to approximately 2 millimeters. In more particular embodiments, the liner curtain 14A, 14B through which ultraviolet light is to be emitted has a thickness less than that of the other liner curtain 14A, 14B. In embodiments, a thickness of a liner curtain 14A, 14B can be thinned in an area corresponding to an ultraviolet light source 24, from which ultraviolet light is to be emitted.

In embodiments, the shower curtain 12 and ultraviolet light sources 24 can be configured to emit ultraviolet light from only one side of the shower curtain 12. In this case, a liner curtain 14A forming the opposing side can be fabricated from an ultraviolet absorbing or ultraviolet reflecting material. However, it is understood that the ultraviolet light sources 24 can be oriented to emit ultraviolet radiation only in a direction away from the liner curtain 14A. In embodiments, the liner curtain 14A forming the opposing side can be fabricated from standard shower curtain material, such as polyethylene vinyl acetate (PEVA), ethylene vinyl acetate (EVA), and/or the like.

In embodiments, some or all of the shower curtain 12 and ultraviolet light sources 24 can be configured to emit ultraviolet light out both sides of the shower curtain 12. In more particular embodiments, a bottom portion of the shower curtain 12 can be configured to emit ultraviolet light from both sides so as to treat a surface of the bathtub 2 located adjacent to the shower curtain 12 when it is located within the bathtub 2.

In embodiments, both liner curtains 14A, 14B can be fabricated from standard shower curtain materials, with the ultraviolet light sources 24 embedded therein. In this case, one or both liner curtains 14A, 14B can include openings from which ultraviolet light can be emitted. For example, an opening can be sized to accommodate a dome of a light emitting diode. In this case, the liner curtains 14A, 14B can be sealed together around the edges of the LED dome to maintain a waterproof barrier, while allowing ultraviolet light to irradiate the intended areas. In embodiments, the openings can be sealed with an ultraviolet transparent sealant.

As illustrated in FIG. 3B, the shower curtain 12 also can comprise connectivity structures 29A embedded therein. In embodiments, the connectivity structures 29A can comprise wiring, which provides power to the ultraviolet light sources 24. In embodiments, the connectivity structures 29A can comprise light guiding structures, which enable ultraviolet light to travel through the curtain 12 before being emitted from an ultraviolet light source 24. In more particular embodiments, the ultraviolet light sources 24 can be connected in a matrix or a series-parallel combination.

In embodiments, the shower curtain 12 also can include an interface 28, which enables selective connection via a connectivity structure 29B to a power and control housing 20, which can be mounted, for example, on a wall in a location near the shower curtain 12. In embodiments, the interface 28 and connectivity structure 29B can comprise any type of interface, which can enable power, control signals, ultraviolet radiation, and/or the like, to be transmitted to or from the various components embedded in the shower curtain 12. In embodiments, the shower curtain 12 can comprise two interfaces 28, one on each side of the shower curtain 12, which can enable selective connection via the connectivity structure 29B to a control housing 20 that can be mounted on either side of the shower curtain 12. In this case, the shower curtain 12 can include a cover that can be secured to the unused interface 28 to prevent moisture and other contaminants from entering the unused interface 28.

In embodiments, the interface 28 can be connected to the power and control housing 20 via a wired connectivity structure 29B, such as a wire connecting a pair of USB ports, or the like. Such a configuration can enable selective location and mounting of the power and control housing 20 to a wall. In embodiments, the interface 28 can be connected to the power and control housing 20 using a connectivity structure 29B that secures the interface 28 to the power and control housing 20, e.g., a plug, a magnetic connection, etc. In this configuration, the end of the shower curtain 12 can be secured adjacent to the wall to prevent a user from inadvertently moving and disconnecting or breaking the connectivity structure 29B. In embodiments, an end of the shower curtain 12 can be secured to a wall using a solution, such as a magnetic mount, which is implemented apart from the power and control housing 20 and the connectivity structure 29B.

The power and control housing 20 can include various additional components of the ultraviolet treatment system 10. To this extent, the power and control housing 20 can include one or more output devices, such as one or more visible light sources 22, which can provide information regarding an operating status of the ultraviolet treatment system 10 for use by an individual. In embodiments, the output devices can provide information on an operability of the system, an operating status of the system, an alert condition, and/or the like. In embodiments, the output devices can include other types of output devices, such as a speaker for emitting an audible output, a wireless communication device for sending output for processing on another computing device, and/or the like.

Additionally, the power and control housing 20 is illustrated as including a set of sensors 26. In embodiments, the set of sensors 26 can include one or more one or more motion sensors and/or proximity sensors, which can detect a presence of a person in a vicinity of the bathtub 2. In embodiments, the set of sensors 26 can include one or more of a light sensor, a temperature sensor, a humidity sensor, and/or the like. Data acquired by the set of sensors 26 can be used in operation of the ultraviolet light sources 24. It is understood that these sensors are only illustrative of various sensors that can be implemented as part of an ultraviolet treatment system described herein.

The power and control housing 20 also is shown including a computing unit 30, which can be configured to control operation of the ultraviolet light sources 24, e.g., using data received from the sensor(s) 26. The computing unit 30 can comprise any type of computing unit capable of controlling operation of one or more input and/or output devices in order to treatment an area with ultraviolet light. Embodiments of the computing unit 30 can include a programmable computer (e.g., including one or more processors, memory, and I/O interfaces) executing program code installed thereon, one or more special purpose microprocessors, and/or the like.

In embodiments, the computing unit 30 comprises one or more real-time controllers (RTCs). The RTC(s) can enable programmable scheduling of UV treatment cycles based on, for example, user preferences, usage patterns, etc. For example, UV treatment can be scheduled during periods of low human activity, such as late at night or early morning, e.g., to maximize effectiveness while minimizing disruption to users. UV treatment also can be scheduled to occur only after use of the bathtub 2, after a predetermined number of uses of the bathtub 2, after a predetermined time from a previous treatment, and/or the like. Embodiments of the treatment system 10 can incorporate scheduled treatment periods to provide for energy reduction, extended operating lifetime of the illumination system, maintaining optimal operating temperatures, etc. However, it is understood that embodiments can provide persistent operation, which can ensure continuous disinfection, e.g., in environments where microbial contamination is a persistent concern.

The computing unit 30 can be configured to operate the ultraviolet light sources 24 using any solution. For example, after use of the bathtub 2, the computing unit 30 can turn on the ultraviolet light sources 24 for a predetermined period of time to deliver a desired dose of ultraviolet light to surfaces of and around the bathtub 2. In embodiments, the computing unit 30 can wait for a period of time after use of the bathtub 2 prior to turning on the ultraviolet light sources 24, e.g., to provide time for any moisture to at least partially dry. In embodiments, the computing unit 30 can obtain humidity level information from a humidity sensor indicating use of the bathtub 2. In response, the computing unit 30 can schedule an ultraviolet treatment of the bathtub 2 to be performed, e.g., a predetermined amount of time thereafter, after the detected humidity level drops below a predetermined threshold, and/or the like.

The computing unit 30 can operate the ultraviolet light source 24 in any manner to deliver a desired dose of ultraviolet light to the surfaces as part of a treatment thereof. For example, the computing unit 30 can operate the ultraviolet light source 24 to generate ultraviolet light in a pulsed manner, continuously, for multiple predetermined periods of time separated by predetermined periods of time when the ultraviolet light sources are off, and/or the like.

Inclusion of RTCs or similar computing units, can enable the timing of UV-based treatment based on user preferences, usage patterns, and/or the like. Embodiments of the RTCs can be programmable with predefined schedules and/or user-configurable settings, which can provide automated and/or customizable control over treatment cycles. While embodiments can provide alternative control methods, such as manual switches, remote control options, etc., inclusion of RTCs or similar computing units can provide a more efficient and user-friendly solution for managing disinfection operations.

Additionally, the power and control housing 20 can include a power component 32. The power component 32 can comprise any type of power source. For example, the power component 32 can comprise one or more batteries, an electrical plug and transformer for obtaining power from an electrical receptacle, and/or the like. Regardless, the power component 32 can be configured to provide sufficient power to operate the remaining components of the ultraviolet treatment system 10. In embodiments, the power component 32 can comprise a rechargeable battery, which can be recharged using a wireless charging solution, using ambient light, and/or the like.

In embodiments, the power and control housing 20 can include a more complicated user interface device, such as a touchscreen, a display with multiple input controls, etc. In this case, the power and control housing 20 can enable communication of additional information regarding the ultraviolet treatment system 10, user selection of additional settings or commands for the operation of the ultraviolet treatment system 10, and/or the like. In embodiments, such information can include an identification of one or more malfunctioning components, a time since a last treatment, a treatment history, a battery life, a current temperature, humidity, etc., and/or the like. In embodiments, such settings can include a duration for a treatment, a time frame within which treatments should be performed, a request to perform a self-check, calibration operation, etc. In embodiments, such functionality can alternatively or additionally be implemented via an application executing on an external computing device, such as an app executing on a mobile telephone device, which communicates with the computing unit 30 using a wireless communications solution.

As described herein, the ultraviolet treatment system 10 can be configured to treat surfaces of the bathtub 2 and/or located near the bathtub 2 in order to prevent, suppress, etc., mold growth and the like. In embodiments, the ultraviolet treatment system 10 can be configured to treat a surface of the shower curtain 12 itself, which also may be susceptible to such growth.

In embodiments, a surface to be illuminated by an ultraviolet light source 24 can be treated in order to prevent the surface from being damaged by the ultraviolet light and/or with a material that is used in conjunction with the ultraviolet light. For example, a surface can be coated with photocatalyst, such as a titanium dioxide coating, which can work in conjunction with ultraviolet A light in order to treat an area. Additionally, ultraviolet stabilizers or blockers can be used to absorb or reflect UV rays, thereby protecting surfaces from UV-induced degradation like color fading or material weakening. Selection of an appropriate coating depends on factors like application specifics and environmental conditions. Embodiments can use UV-resistant sealants and/or photocatalytic sealants. UV-resistant sealants are formulated to withstand UV exposure and maintain their protective properties.

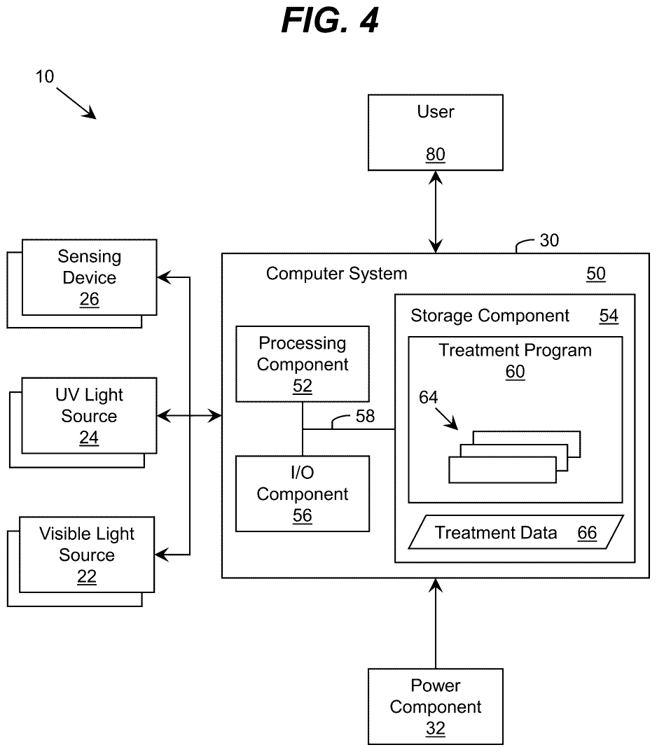

FIG. 4 shows a schematic of an illustrative treatment system 10 according to an embodiment. To this extent, the treatment system 10 includes a computing unit 30, which is implemented as a computer system 50 that can perform a process described herein in order to treat an area with ultraviolet radiation. In particular, the computer system 50 is shown including a treatment program 60, which makes the computer system 50 operable to treat an area by performing a process described herein.

The computer system 50 is schematically illustrated as including a processing component 52 (e.g., one or more processors), a storage component 54 (e.g., a storage hierarchy), an input/output (I/O) component 56 (e.g., one or more I/O interfaces and/or devices), and a communications pathway 58. In general, the processing component 52 executes program code, such as the treatment program 60, which is at least partially fixed in storage component 54. While executing program code, the processing component 52 can process data, which can result in reading and/or writing transformed data from/to the storage component 54 and/or the I/O component 56 for further processing. The pathway 58 provides a communications link between each of the components in the computer system 50.

The I/O component 56 can comprise one or more human I/O devices, which enable a human user 80 to interact with the computer system 50 and/or one or more communications devices to enable a system user 80 (e.g., a portable computing device of a human user, such as a mobile phone executing an app) to communicate with the computer system 50 using any type of communications link. To this extent, the treatment program 60 can manage a set of interfaces (e.g., graphical user interface(s), application program interface, and/or the like) that enable human and/or system users 80 to interact with the treatment program 60 and the treatment data 64, etc. Furthermore, the treatment program 60 can manage (e.g., store, retrieve, create, manipulate, organize, present, etc.) the treatment data 64 using any solution.

In any event, the computer system 50 can comprise one or more general purpose computing articles of manufacture (e.g., computing devices) capable of executing program code, such as the treatment program 60, installed thereon. As used herein, it is understood that “program code” means any collection of instructions, in any language, code or notation, that cause a computing device having an information processing capability to perform a particular action either directly or after any combination of the following: (a) conversion to another language, code or notation; (b) reproduction in a different material form; and/or (c) decompression. To this extent, the treatment program 60 can be embodied as any combination of system software and/or application software.

Furthermore, the treatment program 60 can be implemented using a set of modules 64. In this case, a module 64 can enable the computer system 50 to perform a set of tasks used by the treatment program 60, and can be separately developed and/or implemented apart from other portions of the treatment program 60. As used herein, the term “component” means any configuration of hardware, with or without software, which implements the functionality described in conjunction therewith using any solution, while the term “module” means program code that enables a computer system 50 to implement the actions described in conjunction therewith using any solution. Regardless, it is understood that two or more components, modules, and/or systems may share some/all of their respective hardware and/or software. Furthermore, it is understood that some of the functionality discussed herein may not be implemented or additional functionality may be included as part of the computer system 50.

When the computer system 50 comprises multiple computing devices, each computing device can have only a portion of the treatment program 60 fixed thereon (e.g., one or more modules 64). In embodiments, the computer system 50 can comprise a computing unit located within a component of the faucet and a portable computing unit, such as a mobile phone, which is executing an app installed thereon for enabling a user 80 to monitor, evaluate, manage, and/or the like, the illumination system.

However, it is understood that the computer system 50 and the treatment program 60 are only representative of various possible equivalent computer systems that may perform a process described herein. To this extent, in other embodiments, the functionality provided by the computer system 50 and the treatment program 60 can be at least partially implemented by one or more computing devices that include any combination of general and/or specific purpose hardware with or without program code. In each embodiment, the hardware and program code, if included, can be created using standard engineering and programming techniques, respectively.

Regardless, when the computer system 50 includes multiple computing devices, the computing devices can communicate over any type of communications link. Furthermore, while performing a process described herein, the computer system 50 can communicate with one or more other computer systems using any type of communications link. In either case, the communications link can comprise any combination of various types of optical fiber, wired, and/or wireless links; comprise any combination of one or more types of networks; and/or utilize any combination of various types of transmission techniques and protocols. In embodiments, the computer system 50 can communicate with a user 80 using a Bluetooth or similar wireless communications system.

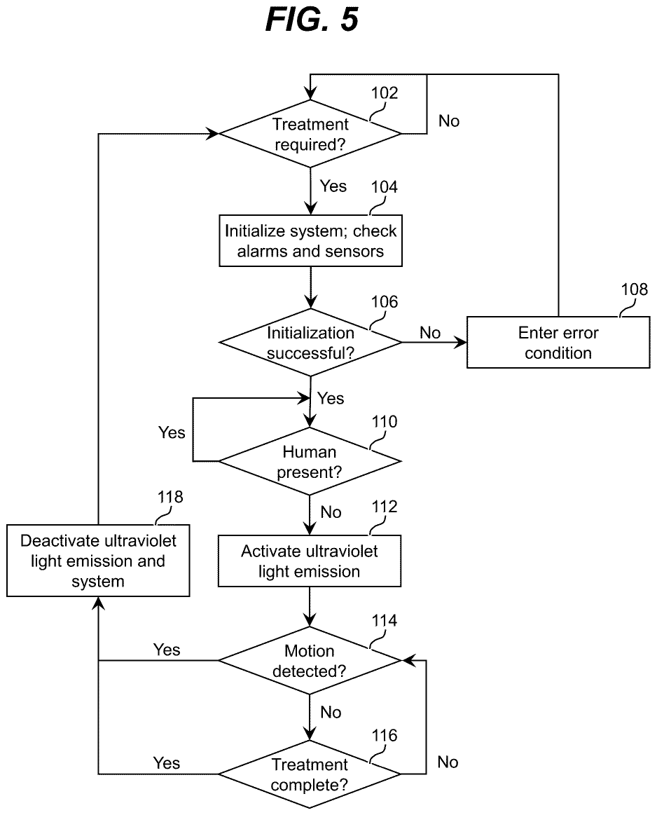

As discussed herein, the computing unit 30 can operate the various I/O devices 22, 24, 26 in order to safely and effectively treat a tub area located around a shower curtain 12. FIG. 5 shows an illustrative process, which can be implemented by the computing unit 30, for treating an area around a shower curtain 12 according to embodiments.

In action 102, the computing unit 30 can determine whether a treatment is required. For example, a treatment may be required after an initial startup, after a selected period of time, at a specified time, after an event (e.g., humidity level above a threshold), in response to a user request, etc. When no treatment is required, the computing unit 30 can remain in action 102 and can enter a power saving mode for a period of time before checking again or receiving a request.

When a treatment is required, in action 104, the computing unit 30 can initialize the various components of the system, check the operation of the alarms and sensors, and/or the like. In action 106, the computing unit 30 can determine if the initialization was successful. If not, the computing unit 30 can enter an error condition in action 108 and halt further operation as described further herein.

When the initialization is successful, in action 110, the computing unit 30 can determine whether any human (or other animal) has been indicated as being present by the sensor data. If so, the computing unit 30 can pause further action until the sensor data indicates that the human is no longer present. Once no one is present, in action 112, the computing unit 30 can activate the ultraviolet light emission in order to treat the tub surfaces located around the shower curtain.

While treatment is being performed, the computing unit 30 can monitor the sensor data for any motion in action 114 and monitor the progress of the treatment in action 116 to determine whether it has completed (e.g., a specified dose has been delivered, a specified duration has completed, and/or the like). In response to motion being detected or the treatment competing, the computing unit 30 can deactivate the ultraviolet light emission and other system components in action 118 and return to action 102. In the event that a treatment is shortened by the detection of motion, the computing unit 30 can determine that treatment is still required and proceed as discussed herein, e.g., after a delay. Otherwise, the computing unit 30 can remain in action 102 until a subsequent treatment is required.

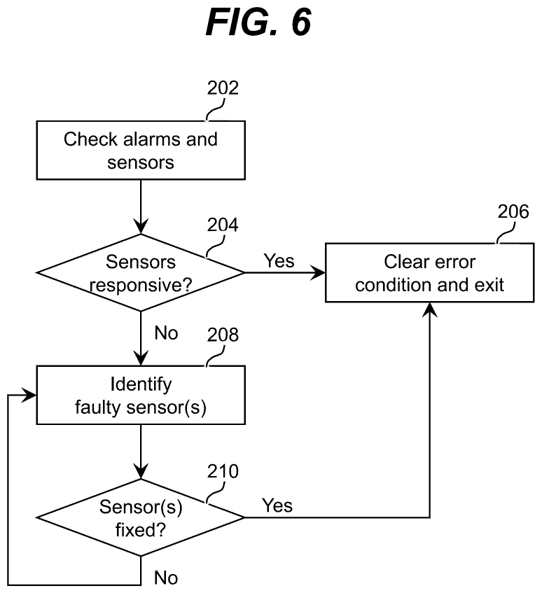

FIG. 6 shows an illustrative process for handling an error condition, which can be implemented by the computing unit 30, according to embodiments. As discussed herein, the computing unit 30 can periodically check the operation of the various components of the system in order to ensure safe and effective emission of the ultraviolet radiation.

In response to an error (e.g., which can occur during initialization), in action 202, the computing unit 30 can check the various alarms and sensors to determine which, if any, are not functioning properly using any solution. For example, the computing unit 30 can execute a system test, which isolates and evaluates the operability of each individual component to determine if any are not functioning properly. In action 204, the computing unit 30 can determine whether any of the sensors was not responsive during the check. If all sensors are responsive, in action 206, the computing unit 30 can clear the error condition and exit from the error condition.

Otherwise, in action 208, the computing unit 30 can identify the faulty sensor(s). Such identification can include generating an error message for presentation to a user. In action 210, the computing unit 30 can determine whether the sensor(s) have been fixed (e.g., by detecting a signal, by receiving an indication from a user, etc.). If so, the computing unit 30 can clear the error condition and exit back to the main process.

In embodiments, in response to an indication that one or more sensors or ultraviolet light sources located on the shower curtain 12 is not operating properly, a user can replace the entire shower curtain 12 with a new shower curtain, while keeping the remaining components. In this manner, the system can be maintained without requiring any complex disassembly or operations. In embodiments, multiple shower curtain configurations can be provided, and the computing unit 30 can automatically determine which shower curtain configuration is installed and adjust operation accordingly. In embodiments, shower curtain configurations with additional functionality can be implemented and the computing unit 30 can be configured to support the additional functionality through a software upgrade, which can be performed over the Internet or the like.

As used herein, unless otherwise noted, the term “set” means one or more (i.e., at least one) and the phrase “any solution” means any now known or later developed solution. The singular forms “a,” “an,” and “the” include the plural forms as well, unless the context clearly indicates otherwise. Additionally, the terms “comprises,” “includes,” “has,” and related forms of each, when used in this specification, specify the presence of stated features, but do not preclude the presence or addition of one or more other features and/or groups thereof.

As also used herein, a layer is a transparent layer when the layer allows at least ten percent of radiation having a target wavelength, which is radiated at a normal incidence to an interface of the layer, to pass there through. Furthermore, as used herein, a layer is a reflective layer when the layer reflects at least ten percent of radiation having a target wavelength, which is radiated at a normal incidence to an interface of the layer. In an embodiment, the target wavelength of the radiation corresponds to a wavelength of radiation emitted or sensed (e.g., peak wavelength+/−five nanometers) by an active region of an optoelectronic device during operation of the device. For a given layer, the wavelength can be measured in a material of consideration and can depend on a refractive index of the material.

It is understood that, unless otherwise specified, each value is approximate and each range of values included herein is inclusive of the end values defining the range. Terms of degree such as “generally,” “substantially,” “about,” and “approximately” as used herein mean a reasonable amount of deviation of the modified term such that the end result is not significantly changed. For example, these terms can be construed as including a deviation of at least +/−0.5% of the modified term if this deviation would not negate the meaning of the word it modifies. In a more particular example, the term “approximately” is inclusive of values within +/−ten percent of the stated value, while the term “substantially” is inclusive of values within +/−five percent of the stated value when these deviations would not negate the meaning of the word each term modifies. Unless otherwise stated, two values are “similar” when the amount of deviation between the two values does not significantly change the result. In a more particular example, two values are similar when the smaller value is within +/−twenty-five percent of the larger value. A value, y, is on the order of a stated value, x, when the value y satisfies the formula 0.1x≤y≤10x.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to an individual in the art are included within the scope of the invention as defined by the accompanying claims.

What is claimed is:

1. A shower curtain comprising:
a first liner curtain fabricated from a first water-resistant material;
a second liner curtain fabricated from second water-resistant material, wherein the second liner curtain is secured to the first liner curtain; and
a plurality of ultraviolet light sources embedded between the first and second liner curtains,
wherein at least one of the first water-resistant material or the second water-resistant material is an ultraviolet transparent material, and
wherein each of the plurality of ultraviolet light sources is configured to emit ultraviolet light through a corresponding location of the ultraviolet transparent material.

2. The shower curtain of claim 1, wherein at least one of the first water-resistant material or the second water-resistant material is and/or is coated with a mold and/or mildew-resistant material.

3. The shower curtain of claim 1, wherein one of the first liner curtain or the second liner curtain is not an ultraviolet transparent material and has a thickness greater than a thickness of the other of the first liner curtain or the second liner curtain.

4. The shower curtain of claim 1, wherein the plurality of ultraviolet light sources is configured to emit ultraviolet light from a plurality of locations spaced along substantially all of one of the first liner curtain or the second liner curtain, and the plurality of ultraviolet light sources is configured to emit ultraviolet light from a plurality of locations spaced along only a bottom portion of the other of the first liner curtain or the second liner curtain.

5. The shower curtain of claim 1, further comprising a plurality of connectivity structures embedded between the first liner curtain and the second liner curtain, wherein each connectivity structure connects at least one of the ultraviolet light sources to at least one of a power source or an ultraviolet light emitting device.

6. The shower curtain of claim 5, further comprising an interface configured to selectively connect the plurality of connectivity structures to at least one of an external power source or an external ultraviolet light emitting device.

7. An ultraviolet treatment system comprising:
a power and control component; and
a shower curtain, the shower curtain comprising:
at least one interface configured for selective connection to the power and control component;
a first liner curtain fabricated from a first water-resistant material;
a second liner curtain fabricated from second water-resistant material,
wherein the second liner curtain is secured to the first liner curtain;
a plurality of ultraviolet light sources embedded between the first and second liner curtains; and
a plurality of connectivity structures embedded between the first liner curtain and the second liner curtain,
wherein each connectivity structure connects at least one of the plurality of ultraviolet light sources to the at least one interface, and
wherein each of the plurality of ultraviolet light sources is configured to emit ultraviolet light through a location of at least one of the first liner curtain or the second liner curtain, and
wherein the power and control component is configured to operate the ultraviolet light sources to treat at least one surface located around the shower curtain.

8. The ultraviolet treatment system of claim 7, wherein at least one of the first water-resistant material or the second water-resistant material is an ultraviolet transparent material.

9. The ultraviolet treatment system of claim 7, wherein at least one of the first water-resistant material or the second water-resistant material is and/or is coated with a mold and/or mildew-resistant material.

10. The ultraviolet treatment system of claim 7, wherein one of the first liner curtain or the second liner curtain is not an ultraviolet transparent material and has a thickness greater than a thickness of the other of the first liner curtain or the second liner curtain.

11. The ultraviolet treatment system of claim 7, wherein the plurality of ultraviolet light sources is configured to emit ultraviolet light from a plurality of locations spaced along substantially all of one of the first liner curtain or the second liner curtain, and the plurality of ultraviolet light sources is configured to emit ultraviolet light from a plurality of locations spaced along only a bottom portion of the other of the first liner curtain or the second liner curtain.

12. The ultraviolet treatment system of claim 7, wherein the power and control housing includes at least one output device configured to provide information to a user regarding an operating status of the ultraviolet treatment system.

13. The ultraviolet treatment system of claim 7, wherein the power and control housing includes at least one computing unit configured to operate the ultraviolet light sources to treat at least one surface located around the shower curtain.

14. The ultraviolet treatment system of claim 13, wherein the power and control housing further includes at least one sensing device configured to detect a presence of a person in a vicinity of the shower curtain, wherein the at least one computing unit is configured to turn off the ultraviolet light sources in response to detection of the presence of a person by the at least one sensing device.

15. The ultraviolet treatment system of claim 13, wherein the power and control housing includes at least one wireless communication device configured to enable communication between the at least one computing unit and an application executing on an external computing device.

16. An ultraviolet treatment system comprising:
a power and control component comprising:
at least one computing unit; and
at least one output device configured to provide information to a user regarding an operating status of the ultraviolet treatment system; and
a shower curtain, the shower curtain comprising:
at least one interface configured for selective connection to the power and control component;
a first liner curtain fabricated from a first water-resistant material;
a second liner curtain fabricated from second water-resistant material,
wherein the second liner curtain is secured to the first liner curtain;
a plurality of ultraviolet light sources embedded between the first and second liner curtains; and
a plurality of connectivity structures embedded between the first liner curtain and the second liner curtain,
wherein each connectivity structure connects at least one of the plurality of ultraviolet light sources to the at least one interface, and
wherein each of the plurality of ultraviolet light sources is configured to emit ultraviolet light through a location of at least one of the first liner curtain or the second liner curtain; and
wherein the at least one computing unit is configured to operate the ultraviolet light sources to treat at least one surface located around the shower curtain.

17. The ultraviolet treatment system of claim 16, wherein the power and control housing further includes at least one sensing device configured to detect a presence of a person in a vicinity of the shower curtain, wherein the at least one computing unit is configured to turn off the ultraviolet light sources in response to detection of the presence of a person by the at least one sensing device.

18. The ultraviolet treatment system of claim 16, wherein the power and control housing includes at least one wireless communication device configured to enable communication between the at least one computing unit and an application executing on an external computing device.

19. The ultraviolet treatment system of claim 16, wherein at least one of the first water-resistant material or the second water-resistant material is an ultraviolet transparent material.

20. The ultraviolet treatment system of claim 16, wherein the plurality of ultraviolet light sources is configured to emit ultraviolet light from a plurality of locations spaced along substantially all of one of the first liner curtain or the second liner curtain.
